(19) United States
(12) Patent Application Publication
(10) Pub. No.: US 2014/0134711 A1
(43) Pub. Date: May 15, 2014

(54) MICROSTRUCTURED MEASURING CHIP FOR OPTICALLY MEASURING PROPERTIES OF ARTIFICIAL OR BIOLOGICAL MEMBRANES, AND METHOD FOR ITS PRODUCTION THEREOF

(71) Applicant: Guido Boese, Muenster (DE)

(72) Inventor: Guido Boese, Muenster (DE)

(73) Assignee: NANOSPOT GMBH, Muenster (DE)

(21) Appl. No.: 13/809,720

(22) PCT Filed: Mar. 10, 2011
(86) PCT No.: PCT/DE2011/075041

§ 371 Date: Sep. 11, 2013

(30) Foreign Application Priority Data
Jul. 12, 2010 (DE) 10 2010 036 344.8

(51) Int. Cl. G01N 33/483; G01N 21/64

(52) U.S. Cl. CPC G01N 33/4833; G01N 21/6486
USPC 435/288.7; 422/69; 422/82.05; 422/561; 216/2; 427/2.1; 156/256

(57) ABSTRACT

A microstructured measurement chip (1) for optical measurement of properties of artificial or biological membranes (40), having a lower, translucent support layer (10) and at least one non-translucent main layer (20) disposed on top of the former, which layer has depressions (30) configured as measurement chambers, having an upper opening (25) and one or multiple inner side walls (26). In order to improve the measurement chip (1) in such a manner that biological systems can be measured with greater measurement accuracy and higher throughput, it is proposed that the side wall or the side walls (26) of the measurement chambers (30) have depressions (27) and/or elevations (28). The invention furthermore relates to a holder (200) for the measurement chips (1) as well as to a method for the production of the measurement chips (1) from a silicon wafer (300).

The invention relates to a microstructured measurement chip for optical measurement of properties of artificial or biological membranes, having a lower, translucent support layer and at least one non-translucent main layer disposed on top of the former, which layer has depressions configured as measurement chambers, having an upper opening and an inner side wall or multiple inner side walls. The invention furthermore relates to a method for the production of the measurement chip and to a holder for these measurement chips.

Biological membranes not only separate cells from an external medium, but also separate individual cell compartments within the cells. Membrane transport systems. Such as, for example, transport proteins, channel proteins, secretory systems, and membrane pores selectively allow and control passage of substances through these membranes, by means of changing the membrane permeability. Receptors, in contrast, convey signals, such as, for example, an extracellular ligand bond, which leads to secondary processes on the intracellular level.

Dysfunctions of the transporters and channels are responsible for numerous widespread diseases. Among the 100 medications most frequently sold in the USA in 2004, the most frequent ones were those whose mechanism of action is based on membrane transport systems. At least 1,302 such medications are present in the portfolios of 326 companies worldwide, specifically those that have been introduced as well as those that are still in development. In total, at present more than 100 membrane transport systems are being researched by pharmaceutical companies, which shows what great economic significance they have.

Methods and devices with which the properties such as the transport rates of specific substrate molecules through membrane transport systems and the influence of active substance candidates can be evaluated are needed for the development of new active substances. In this connection, membrane transport systems must be characterized automatically, at high throughput, in order to thereby allow finding an active substance candidate by means of a statistically significant detection of a change in the transport rate of a predetermined transport substrate by means of the target protein.

Membrane permeability, for example, is decisive for the availability of active substances in cells, but also in the brain, because the blood-brain barrier must be penetrated for this purpose. In the development of active substances, the availability at the target location is therefore a decisive property of potential active substances.

In contrast, effective cellular secretion is decisive for the production of biopharmaceutical products such as antibodies, proteins, and the like, by means of cultures of producing eukaryotic and prokaryotic cells, such as, for example, mammalian cells, ciliates, yeasts, and bacteria. Because a divergence of the production rates of the individual cells occurs in such cultures, despite the monoclonality that is aimed at, finding and selecting highly producing cells is decisive for the production rate of the culture. The global market for biopharmaceutical products is estimated at 70 billion dollars for 2010.

Membrane receptors play a central role in the occurrence of many diseases that have significance in political economics, such as, for example, allergies, neurological illnesses, depressions, pain, inflammations, diabetes, epilepsy, high blood pressure, or asthma. Among the membrane receptors, a market share of $12.7 billion already occurred for the subgroup of protein kinases alone in 2002, with a predicted increase to $58.6 billion in the year 2010 (Biophoenix Consulting).

Receptor proteins such as G-protein-coupled receptors (abbreviated GPCR) possess extracellular regions for ligand binding, transmembrane regions, and intracellular domains that serve for passing the signal on to cellular signal cascades. For the characterization of the receptor activation, detection of the signal, in other words of the conformation change of the intracellular domains or the formation of the subsequent components of the signal cascade is required.

In turn, electrical measurements can be used for the analysis of transport rates of ions and charged particles. This method is already in use in the higher throughput in biotechnological and pharmaceutical research. However, it is restricted to ions and is therefore used only for the group of the ion channels. The transport of molecules such as amino acids, peptides, sugar compounds, and fatty acids, but also of biological macromolecules such as RNA, DNA, and proteins cannot be measured using electrical methods, or can only be measured indirectly.

In contrast, fluorescence analysis is very well suited for measuring the transport of these molecules. Preliminary advance work for this was performed by an academic group for the transport of biomolecules by means of the nuclear pore complex in nuclear envelopes from Xenopus laevis. It was also used for measuring the transport of calcium ions through the α-hemolysin pore, which was directly inserted into prefinished, artificial lipid membranes, and in this connection folds back from a denatured structure into a functional form. In the publications, translucent polycarbonate filters or polycarbonate structures were used for this purpose, the depressions of which were used for the fluorescence measurement of transport rates by means of confocal laser scanning microscopy. This results in poor optical properties, among other things due to divergences in the indices of refraction of polycarbonate and the measurement buffer.

Other measurement chips having measurement chambers made of translucent material are known, the depressions of which can be covered up by an upper membrane or cells or tissue, and closed measurement chambers are formed in this way, and the transport of substrate molecules by way of the membrane or the secretion from cells into the measurement chambers can be measured. For this purpose, the membrane or the cells are stretched over the measurement chambers in the measurement chip, so that these are closed off and sealed. The measurement chip is suitable for the analysis of permeability through artificial or biological membranes or cells. Substrate molecules can be detected and quantified in the measurement chambers, by means of optical measurements such as fluorescence measurements. In this connection, as few substrate molecules as possible should be excited to fluorescence outside the measurement chambers, in order not to distort the measurement result.

From the patent application US 2003/0174992 A1, a nanostructured measurement chip is known for fluorescence analysis of biochemical processes, having a translucent support and a non-translucent metal layer that lies above it. The metal layer contains a measurement chamber having a much smaller diameter than the wavelength of the excitation light that is radiated in from below, and thereby acts as a so-called zero-mode waveguide. The excitation light therefore does not penetrate into the depressions, but part of the light energy gets into the measurement chambers. No substrate molecules are excited above the measurement chambers. However, such a measurement chip has numerous disadvantages. For example, the volume of the measurement chamber, at a few zeptoliters, is very small, because of its small dimensions, so that these cannot be used for transport processes. Furthermore, only the lower region of the measurement chamber can be used, because excitation takes place only there. As a result, the signal/noise ratio of the measurements is very poor, and a complicated measurement device is required.

It is the task of the invention to make available the a measurement chip with which the properties of membranes or transport systems can be measured using commercially available, usual measurement devices, at great measurement accuracy and high throughput.

This task is accomplished by means of a microstructured measurement chip having a lower, translucent support layer and at least one essentially non-translucent main layer disposed on top of the former, which layer has depressions configured as measurement chambers, having an upper opening and an inner side wall or multiple inner side walls, in that the side wall or the side walls of the measurement chambers have depressions and/or elevations. As a result, the excitation light can penetrate into the measurement chambers, with dimensions above the wavelength, from below, and can generate a high fluorescence signal, but it reaches all the way to the upper opening only in greatly weakened form. In the sense of the invention, an essentially translucent layer is understood to mean that the layer is predominantly permeable to light. An essentially non-translucent layer is understood to mean that this layer is predominantly or completely impermeable to light, by means of absorption or reflection. Light is predominantly understood to mean the visible spectrum of electromagnetic radiation, of about 400 to 700 nm; however, the term is not restricted to this, but rather can also comprise the adjacent spectra of ultraviolet or infrared radiation. For the optical measurements, usual fluorescence microscopes with the non-coherent light of a fluorescent lamp but also laser scanning microscopes can be used. The measurement takes place from below, through the translucent support layer of the measurement chip. For this reason, this layer is permeable to the excitation light of a fluorescent lamp. For example, suitable substrate molecules are excited to fluorescence in the measurement chamber or the measurement chambers, by means of the excitation light. This fluorescence is then measured by means of a suitable camera that is coupled into the optics of the fluorescence microscope, and subsequently evaluated. The measurement accuracy that can be achieved with the measurement chip now depends, to a significant degree, on the extent to which emission light that is produced exclusively by means of fluorescence excitation within the measurement chamber is measured. Specifically, if additional emissions are measured, which were produced outside the measurement chamber, these are interference emissions that worsen or distort the measurement result. For example, substrate molecules can be excited in the membrane or in the measurement fluid above the measurement chambers. In order to reduce these interference emissions, the side wall or the side walls of the measurement chambers are not smooth, but rather have depressions and/or elevations, according to the invention. Smooth, reflective side walls would reflect the interference emissions from above the measurement chambers all the way to the camera, while the depressions and elevations scatter the interference emissions and thus minimize any propagation within the measurement chambers. Furthermore, propagation of the excitation light out of the measurement chambers into the membrane or measurement fluid that lies above them is reduced. In total, an undesirable optical detection of substrate molecules within and above the membrane is minimized, and predominant detection of the emissions of substrate molecules within the measurement chambers is achieved. This is also possible using conventional fluorescence microscopy.

Advantageous embodiments are indicated in the dependent claims and will be explained below.

A further improvement in the measurement accuracy can be achieved if the depressions and elevations alternate, in other words are configured in groove-shaped manner, and the grooves form a corrugated surface structure of the side wall or of the side walls in the direction of the longitudinal axis of the measurement chambers.

In a preferred embodiment, the alternating depressions or elevations or grooves have a periodic distance from one another, in each instance, of 0.1 to 0.6 μm. However, the distance can also amount to several nanometers to multiple micrometers. The depth of the grooves, i.e. the distance between the highest point of the elevations and the lowest point of the depressions, amounts to 20 to 110 nm, but can also amount to as much as several micrometers.

In a preferred embodiment, the measurement chambers have the basic shape of a circular cylinder or a truncated cone, in each instance. Because the measurement chambers are formed by means of depressions in the main layer, they themselves do not have any external shape. In the sense of the invention, the basic shape of the measurement chambers should therefore be understood to mean their hollow volume, which is delimited by the main layer. In this sense, the side wall of a measurement chamber is formed by the mantle surface of a circular cylinder or a truncated cone. In both embodiments, the depressions and elevations that form the grooves run around the side wall, i.e. both the measurement chambers and the grooves are approximately circular in section perpendicular to the longitudinal axis of the measurement chambers. Such a corrugated surface structure can be achieved by means of deep reactive silicon ion etching (DRIE, Bosch process) for deep etching of silicon. This comprises an alternating sequence of etching process and passivation step, and, in this connection, produces such a corrugated or chamber structure of the side walls perpendicular to the etching direction. The shape of and the distances between grooves vary in accordance with the process setting and etching depth. The grooves in the side walls made of a non-transparent, more reflective material, produce scattering of both the excitation light that is radiated in and of the emission light in the measurement chambers, while smooth side walls would tend to allow reflection of light longitudinally through the measurement chambers and thus passing it on in a waveguide. In this way, excitation light radiated in from the underside of the measurement chip is better shielded from an exit out of the upper opening of the measurement chamber. Likewise, interference emissions above the measurement chamber are better shielded from passage out of the bottom of the measurement chamber all the way to the camera, and the measurement accuracy as a whole is further improved.

The lower, translucent support layer consists, for example, of plastic or of glass. It has been shown that borosilicate glass that has been produced using the float process or as a polished wafer is particularly suitable.

The non-translucent, microstructured main layer having the depressions that serve as measurement chambers demonstrates metal, plastic, or silicon. The term silicon also comprises silicon compounds. Silicon has the advantage that known methods from the sector of electronic microchips can be used, in part, for processing, in other words for production of the depressions.

A cover layer, preferably composed of silicon dioxide and/or metal, can be disposed on the main layer. The cover layer then has openings that are disposed above the openings of the measurement chambers. Preferably, an opening in the cover layer is disposed above the opening of a measurement chamber, in each instance. In this connection, the aperture of the openings of the cover layer is smaller than the aperture of the openings of the measurement chambers. In this way, shutters are formed by the cover layer, which can partly shield against excitation light radiated in from the underside of the measurement chip when it exits from the upper opening of the measurement chamber. Likewise, interference emissions above the cover layer are blocked off. In this way, the measurement accuracy is further improved. Another advantage lies in that the embodiment of the measurement chips is suitable for biological membranes having a biologically predetermined transporter density. Because the number of transport proteins per surface area cannot be easily changed in the case of biological membranes, the aperture of the cover layer opening can be selected and optimized, at an unchanged measurement chamber volume, in such a manner that preferably only one or only a few transport proteins lie above the cover layer opening. This allows more precise measurements with an extended measurement period.

The surface of the measurement chip can demonstrate one or more chemically reactive and/or polar coatings, particularly poly-L-lysine and/or propionic acid and/or carboxyl groups and/or lipid derivatives and/or amino-reactive linker molecules, in order to bind artificial or natural membranes to the measurement chip directly or indirectly, covalently or non-covalently.

The side wall or the side walls of the measurement chambers and/or the underside of the main layer that lies on the support layer and/or the underside of the cover layer that faces the measurement chambers and/or the top of the main layer can additionally have a non-translucent coating, in each instance, preferably composed of metal, particularly of gold or titanium. This preferably takes place by means of known PVD methods (abbreviation for “physical vapor deposition”). The coating has multiple advantages. If residual translucence of the main layer exists, this is prevented by the coating. For example, silicon is essentially non-transparent for wavelengths of visible light up to 600 nm. Silicon becomes increasingly more permeable for deep red and infrared light. This would be disruptive if the excitation light or interference emissions lie in this wavelength range. The additional non-translucent coating improves the measurement accuracy in these cases. Another advantage is that in the case of a coating made of gold, this can be contacted and used as an electrode for electrical measurements or excitations. Yet another advantage results in combination with the chemically reactive or polar coating mentioned above. Thiol compounds such as β-mercaptoethanol or mercaptopropionic acid, but also components of a detection system of receptor activation can couple to a coating of gold on the side wall of the measurement chambers, in standardized manner. The layer of silicon or silicon oxide does not bind these, and can therefore be modified selectively by means of silanization. In this way, different modification of the measurement chambers and of the top of the measurement chips is made possible, and this is advantageous for specific measurement tasks.

Because of the fact that the ratio of depth to diameter of the measurement chambers is greater than one, preferably greater than five, and particularly preferably ten to fifty, only essentially the small proportion of the excitation light radiated in parallel to the side wall of the measurement chamber can spread out through the upper opening of the measurement chamber. In contrast, the proportion of the excitation light radiated in not parallel to the side wall is scattered or absorbed on the side wall on its way through the measurement chamber, but excites fluorescence in the lower region of the measurement chambers. This effect is reinforced by a diameter of the measurement chamber that decreases in an upward direction. As a result, the measurement accuracy is additionally improved.

The invention furthermore comprises a holder for the microstructured measurement chip described above. The holder comprises a plate having a top, an underside, and one or more reservoir(s) that can be filled with fluid from the top. The holder has a block-like shape, similar to commercially available microtiter plates, for example, and can also be used in similar manner. In a preferred embodiment, it has standardized dimensions, with regard to width, length and/or height, which fulfill the ANSI standards for microtiter plates or cover glasses. In contrast to commercially available microtiter plates, however, the reservoirs are continuous channels, i.e. they do not have a bottom, at first, but rather a lower opening. Instead, the bottom of a reservoir is formed only by a measurement chip attached to the underside of the plate. All the measurement chips of the holder can also first be glued onto a thin glass support in the size of the holder, and then countersunk into the reservoirs from below, so that the glass support is glued under the holder.

Attachment can take place by means of a water-resistant and watertight adhesive, specifically in such a manner that the measurement chambers face in the direction of the reservoirs. When the reservoirs are filled with measurement fluid, the measurement chambers of the measurement chips are therefore also filled. Preferably, the chip is glued below the lower opening of a reservoir with a UV-curing adhesive or an adhesive film, whereby the adhesive is cured by irradiation with UV light after it has been adjusted. Alternatively, when using an additional glass support, a silicone adhesive, preferably Sylguard 184, has proven to be suitable for gluing the measurement chips onto the glass support. In this manner, the reservoirs form a container for a suitable measurement fluid, for the microstructured measurement chips. The optical measurement takes place from the underside of the holder, through the translucent support layer of the measurement chips, or additionally through the glass support and adhesive as described above.

The invention furthermore comprises a method for the production of microstructured measurement chips, particularly having the characteristics described above. In this connection, a silicon wafer is used as the starting material or as a substrate, as used in microelectronics for the production of integrated circuits. The advantage is that known methods for microstructuring such as photolithography and etching can be used.

A “silicon on insulator wafer” or abbreviated “SOI wafer” is particularly suitable. These SOI wafers are known according to the state of the art and consist of three layers: A lower silicon layer, an upper silicon layer, and a so-called buried layer (the English technical term is “buried layer”) disposed between them, which has electrically insulating properties. This layer consists, for example, of silicon dioxide or silicon nitride. Electronic components that are produced from an SOI wafer, for example integrated circuits, have shorter switching times and lower power consumption, because leakage currents are reduced by the buried layer.

It has surprisingly been shown that the use of an SOI wafer, particularly one having a buried layer of silicon dioxide, has advantages in the production of microstructured measurement chips.

The production method comprises the following steps, in detail:

The measurement chambers are etched into the upper silicon layer, all the way to the buried layer of the silicon wafer. Advantageously, in this connection, the buried layer stops the etching process if an etching agent that selectively attacks and dissolves silicon only is used. An acid or a gas can be used as the etching agent, preferably using the DRIE method (Deep Reactive Ion Etching).

If desired, afterward an additional non-translucent coating, such as titanium and/or gold, is applied to the upper silicon layer.

The upper silicon layer of the silicon wafer with the measurement chambers etched into it is then connected with the support layer, preferably by means of anodic bonding, whereby the previous opening of the measurement chamber becomes its underside, with the support layer as the bottom.

Afterward, the lower silicon layer of the silicon wafer is removed, preferably by means of etching. Advantageously, the buried layer stops the etching process in this method step, as well, if an etching agent that selectively attacks and dissolves only silicon is used.

Then the buried layer is removed, entirely or in part, preferably by means of etching, whereby a special etching agent that attacks and dissolves the buried layer, for example hydrofluoric acid, is used. In the case of partial removal, the buried layer is photolithographically structured selectively at those locations at which it covers the measurement chamber openings. The buried layer then forms the cover layer with the cover layer openings of the measurement chip.

Finally, the individual microstructured measurement chips are sawed out of the silicon wafer, particularly in sizes of 2 by 2 mm to 10 by 10 mm. A particularly preferred size is 2.5 by 2.5 mm. The measurement chips can be used individually or, in particular, glued to the holder described above.

The invention will be described making reference to a drawing as an example, whereby further advantageous details can be derived from the figures of the drawing.

In this connection, functionally equivalent parts are provided with the same reference symbols.

The figures of the drawings show, in detail:

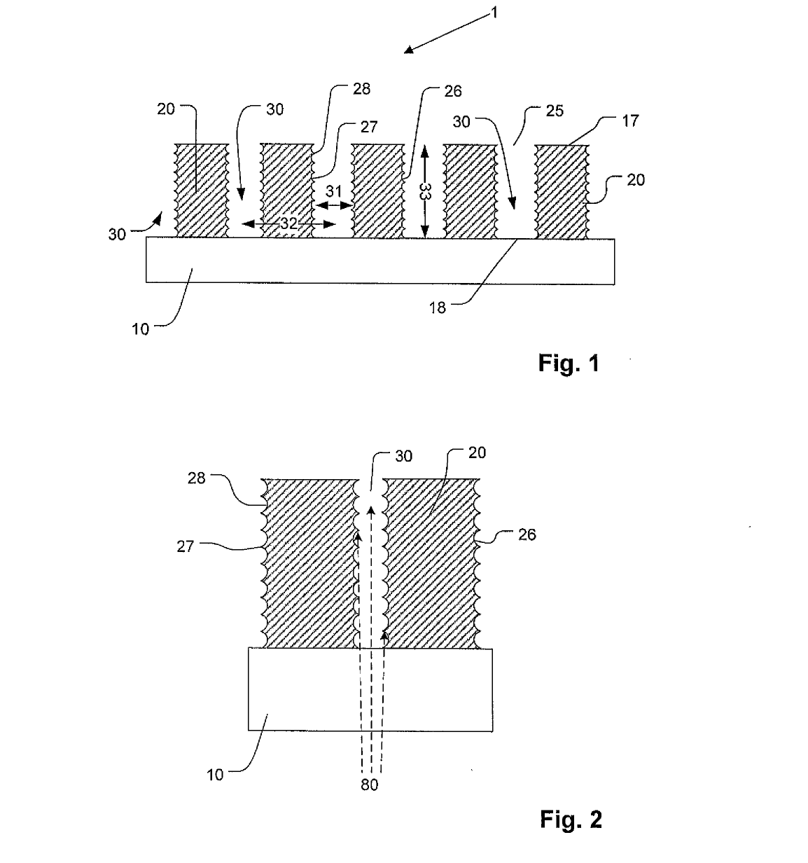

FIG. 1 a vertical section through the measurement chip, in a partial view;

FIG. 2 a detail view of a measurement chamber, in another embodiment of the measurement chip, in vertical section;

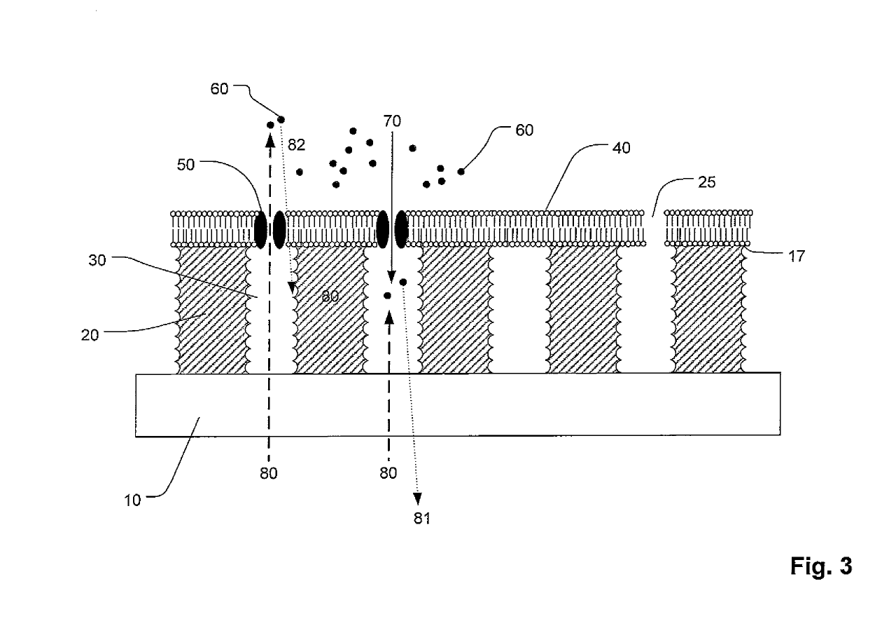

FIG. 3 the measurement chip from FIG. 1, with a lipid membrane;

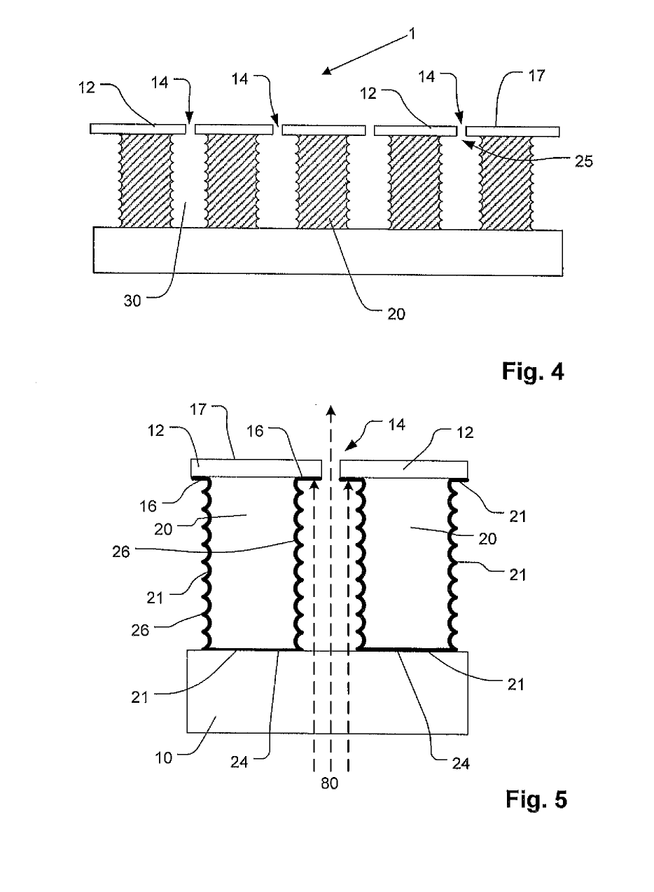

FIG. 4 a vertical section through another embodiment of the measurement chip, with a cover layer, in a partial view;

FIG. 5 a detail view of a measurement chamber with a cover layer and a coating;

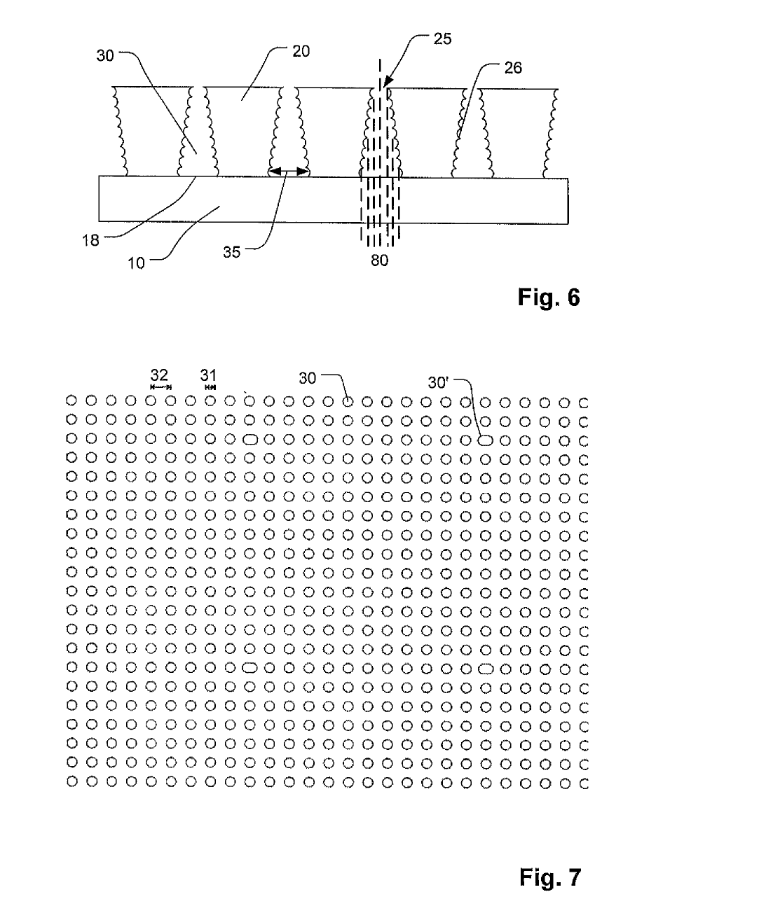

FIG. 6 a vertical section through another embodiment of the measurement chip, with measurement chambers in the shape of truncated cones, in a partial view;

FIG. 7 a top view of the measurement chip, in a partial view;

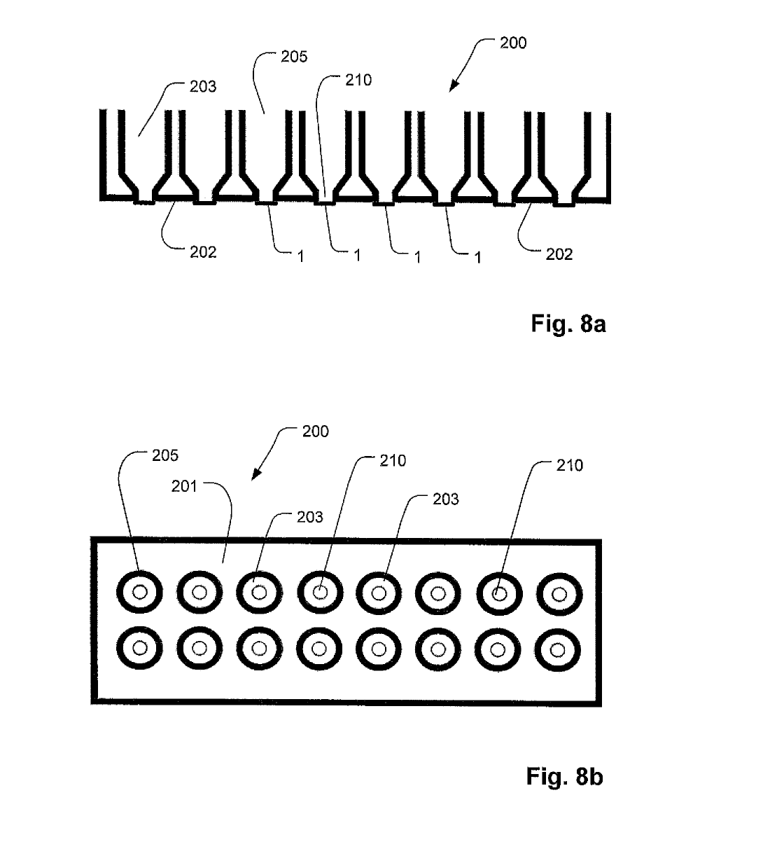

FIG. 8a a vertical section through a holder;

FIG. 8b a top view of the holder from FIG. 8a;

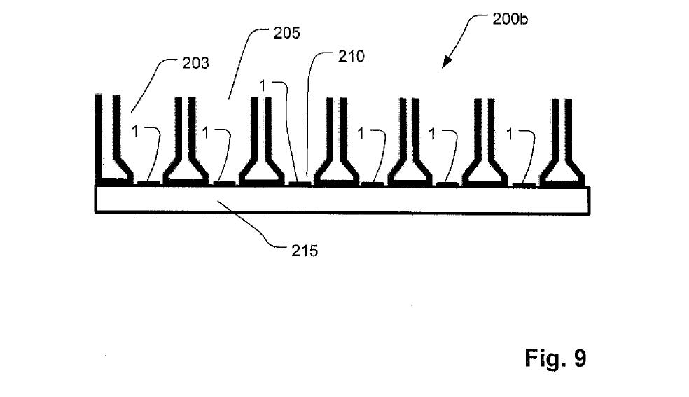

FIG. 9 a vertical section through another embodiment of the holder;

FIG. 10 a vertical section through a known SOI wafer, in a partial view;

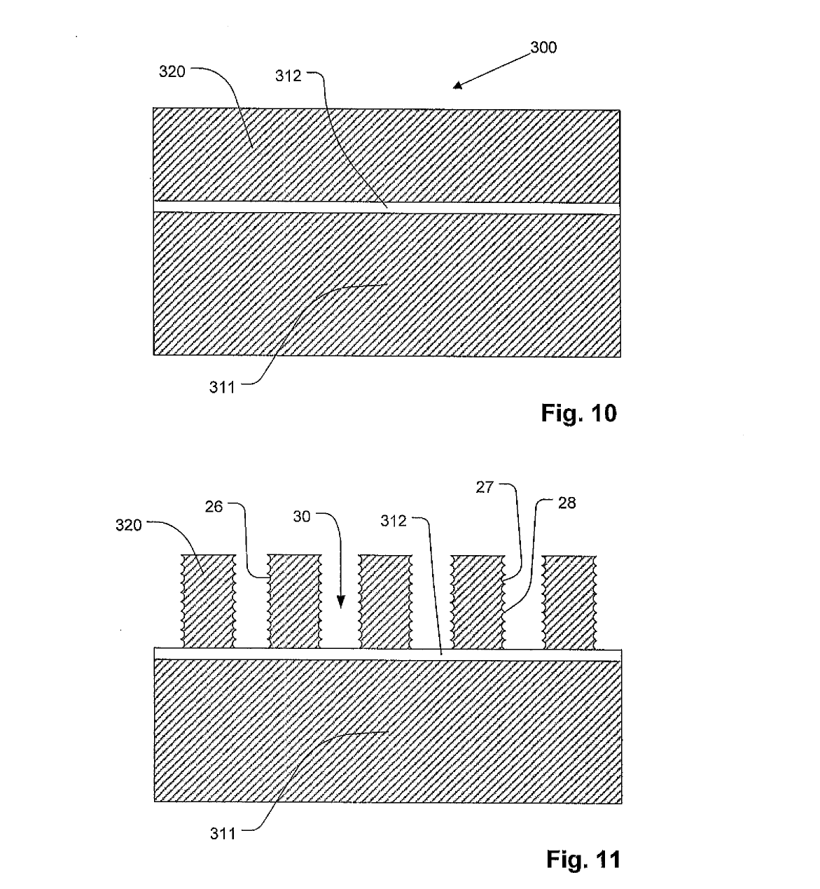

FIG. 11 a vertical section through an SOI wafer as in FIG. 10, with measurement chambers etched in;

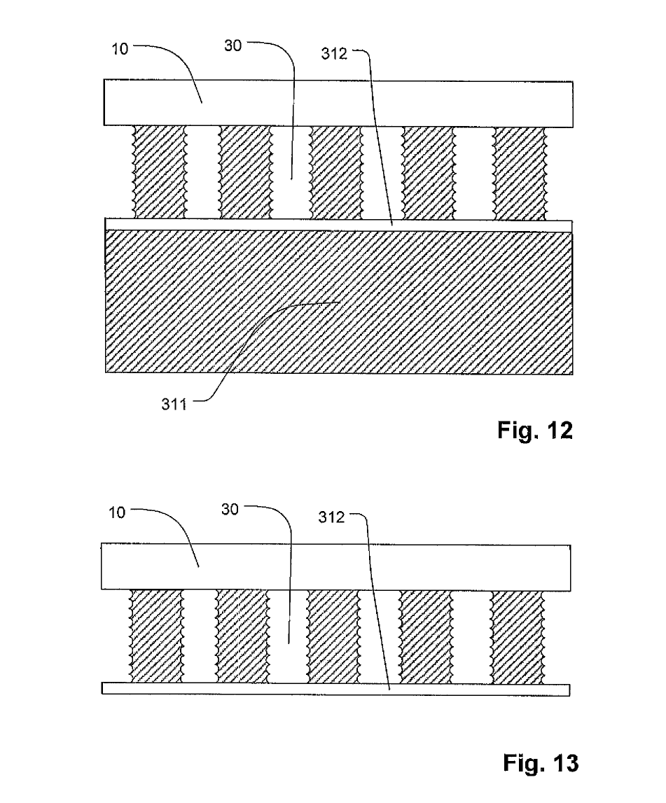

FIG. 12 a vertical section through an SOI wafer as in FIG. 11, with an upper, connected support layer;

FIG. 13 a vertical section through an SOI wafer as in FIG. 12, after removal of the lower silicon layer;

FIG. 14 a vertical section through an SOI wafer as in FIG. 13, after it is turned over;

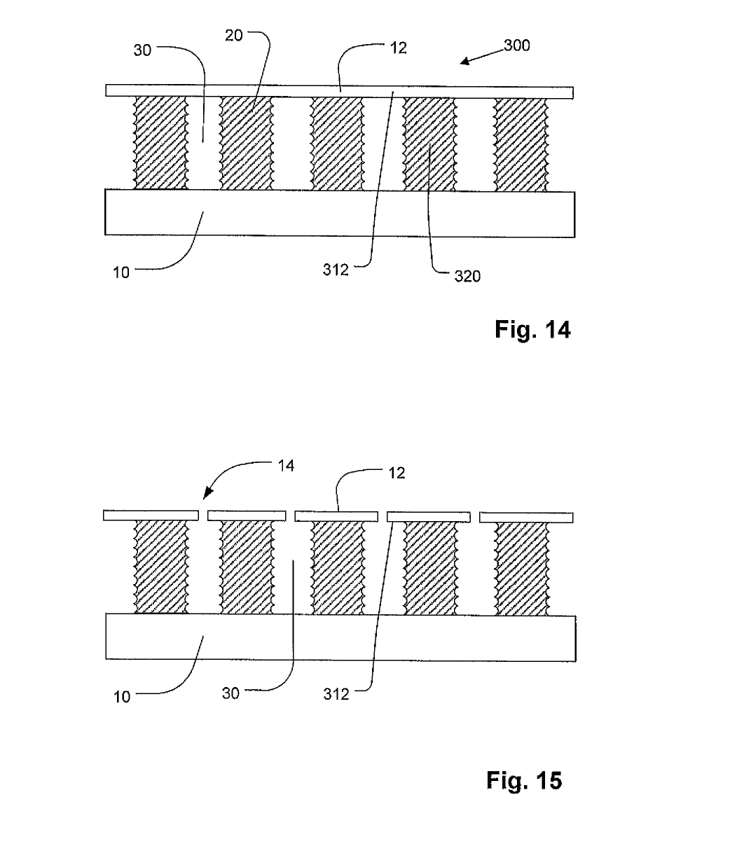

FIG. 15 a vertical section through an SOI wafer as in FIG. 14, after partial removal of the hidden layer.

FIG. 1 shows a partial view of a measurement chip 1 according to the invention, in vertical section. In a top view (not shown), it is square and has a total surface area of 2.5 by 2.5 millimeters, in other words 6.25 square millimeters. The measurement chip 1 consists of layers or materials connected with one another. As a base, it has a lower, translucent support layer 10 made of float or polished borosilicate glass. “Borofloat 30” or “Pyrex” have proven themselves. The thickness of the support layer 10 lies at approximately 140-200 μm, although it can also be thicker or thinner. The support layer 10 is permeable to excitation light 80 or emitted fluorescence light 81. An essentially non-translucent main layer 20 composed of silicon is disposed on the support layer 10, forming the top 17 of the measurement chip 1. For the sake of completeness, it should be noted that the main layer 20 composed of silicon oxidizes externally in air, and therefore a superficial silicon dioxide layer forms, although this has a thickness only in the nanometer range.

The main layer 20 is firmly connected with the support layer 10 by means of anodic bonding. The main layer 20 has continuous depressions in the shape of circular cylinders. The depressions therefore form measurement chambers 30 having a circular-cylindrical hollow volume. The one inner side wall 26 of the measurement chambers 30 is therefore essentially formed by the mantle surface of the circular cylinder, and the circular, upper opening 25 is formed by its cover surface. Because the main layer 20 has continuous depressions, the bottom 18 of each measurement chamber 30 is formed by the top or surface of the translucent support layer 10. The measurement chambers 30 have a depth 33 of 10 to 30 μm, but depths of several nanometers to millimeters are also possible. The diameter 31 of the measurement chambers 30 amounts to about 1 μm, but diameters 31 of several nanometers to a millimeter are also possible. The distance 32 between the longitudinal axes of the individual measurement chambers 30 amounts to 2.5 μm to 4 μm; however, distances 32 of several nanometers up to a millimeter are also possible. The side wall 26 of each measurement chamber 30 is not smooth, but rather demonstrates alternating depressions 27 and elevations 28, which form a corrugated or riffled surface structure. The period of the corrugations lies on the order of 100-600 nm, but can also amount to a few nanometers to several micrometers.

FIG. 2 shows a detail view of a measurement chamber, in another embodiment of the measurement chip 1, in vertical section. In this embodiment, the ratio of depth 22 to diameter 31 of the measurement chambers 30 amounts to about 1 to 10. As a result, only excitation light 80 radiated in essentially parallel to the measurement chamber side wall 26 can spread out through the opening of the measurement chamber. The side walls 26 of the measurement chambers 30 partly have a corrugated surface structure, which is formed by grooves 27, 28 that repeat in the direction of the center axis of the measurement chambers 30. Smooth, reflective side surfaces would reflect light radiated into the measurement chambers 30 further in an upward direction. In contrast, undesirable propagation of excitation light 80 or of interference emissions 82 (not shown, see FIG. 3) within the measurement chambers 30 or out of the measurement chambers 30 is reduced by means of the corrugated surface structure (this is undesirable because only substrate molecules 60 within the measurement chambers 30 are supposed to be excited and detected). This effect is illustrated by the bundle of excitation light 80 of a fluorescent lamp (not shown), which bundle is radiated in straight from below and is not coherent, and is scattered and/or deflected by the depressions 27 and elevations 28. As a result, the propagation of excitation light 80 through the measurement chambers 30 into a membrane and measurement fluid that lies above them (not shown, see FIG. 3) is significantly reduced.

FIG. 3 shows a detail of a measurement chip as in FIG. 1. In addition, a lipid membrane 40 is shown, which is used in measurements with the measurement chip 1. The lipid membrane 40 is applied to the top 17 of the measurement chip 1, so that at least some of the measurement chamber openings 25 are covered and closed off by the lipid membrane. The membrane 40 has been produced from artificial proteoliposomes, which spontaneously fuse with the chip surface when added, and can thereby form the membrane 40. The membrane 40 contains individual transport proteins 50, for example channel proteins, for transporter analyses. Optically detectable substrate molecules 60 are added above the membrane 40; these either fluoresce intrinsically or are covalently marked with a fluorescence dye. The transport 70 of the substrate molecules 60, by means of the transport proteins 50 introduced into the membrane 40, into the measurement chambers 30 of the measurement chip 1 is specific to the transport protein 50 and can be measured by means of detection of the fluorescence in the measurement chambers 30. This permits conclusions concerning specific parameters such as transport rates and permeability, and allows the evaluation of active substance candidates for medications, for example. The measurement takes place in an aqueous medium, i.e. measurement chambers 30, membranes 40, proteins 50, and substrate molecules 60 are surrounded by a measurement fluid (not shown), for example a suitable buffer solution that contains salt. If a holder 200 (not shown, see FIG. 9) is used for the measurement, as it is shown in, then the measurement chip 1 forms the bottom of a reservoir 203 that is filled with measurement fluid above the measurement chip 1.

The measurement takes place, for example, by means of a fluorescence microscope (not shown), which makes available not only a fluorescent lamp or also a laser for the excitation light 80 for excitation of the fluorescence of the substrate molecules 60, as well as enlarging optics. In this connection, the excitation light 80 (shown with a broken line) is radiated into the measurement chambers 30 approximately orthogonally, from below, through the translucent support layer 10, in order to excite the substrate molecules 60 transported into the measurement chamber 30 through the membrane 40, from the top of the measurement chip 1, to fluorescence. The fluorescence emissions 81 (shown with a dotted line) given off by the excited substrate molecules 60 radiate through the translucent support layer 10 from the measurement chamber 30, and are measured by a suitable camera or a detector (not shown) of the fluorescence microscope.

As shown in FIG. 2, undesirable propagation of excitation light 80 out of the measurement chambers 30 is reduced by means of the corrugated surface structure. If a certain residual proportion of the excitation light 80 nevertheless radiates through the measurement chamber 30 and through the membrane 40, then the substrate molecules 60 above the measurement chip 1, in other words outside of the measurement chambers 30, which were not transported into the measurement chambers 30 by way of the membrane 40, are excited, in undesirable manner, and give off interference emissions 82. Propagation of the interference emissions 82 above the measurement chip 1 through the measurement chambers 30 onto a camera is minimized by means of the corrugated surface structure 27, 28. In this way, a significant improvement in measurement accuracy is obtained.

FIG. 4 shows a vertical section through another, preferred embodiment of the measurement chip 1, which essentially corresponds to the one shown in FIG. 1, but has an additional cover layer 12. The cover layer 12 is disposed on the main layer 20. The cover layer has openings 14 that are disposed above the openings 25 of the measurement chambers 30. Preferably, an opening 14 in the cover layer 12 is disposed centered above the opening of a measurement chamber 30, in each instance. In this connection, the aperture of the openings 14 of the cover layer 12 is smaller than the aperture of the openings 25 of the measurement chambers 30. The advantage lies in that the embodiment of the measurement chip 1 shown in FIG. 4 is particularly well suited for biological membranes having a biologically predetermined transporter density. Because the number of transport proteins 50 per surface area cannot be changed, as it can in the case of artificial membranes 40, the aperture of the cover layer opening 14 can be selected and optimized, at an unchanged volume of the measurement chamber 30, in such a manner that preferably only one or only a few transport proteins 50 lie above the cover layer opening 14. The measurement accuracy can be increased by means of the cover layer 12, because fewer substrate molecules per time unit are transported into the measurement chamber 30, and time-resolved measurements can also be conducted, which measurements would not be possible without the cover layer openings 14, because of high transport speeds.

FIG. 5 shows, in vertical section, a detail view of another embodiment of the measurement chip 1 with a measurement chamber 30 having a cover layer 12 of silicon dioxide and an additional non-translucent coating 21 of titanium and/or gold. If gold is used, titanium serves as an adhesion mediator. The components of the measurement chip 1 with metal coating 21 are represented by means of thicker lines in FIG. 6. These are the side walls 26 of the measurement chambers 30, the underside 16 of the cover layer 12 that faces the measurement chamber 30, and the underside 24 of the main layer 20 that lies on the support layer 10.

The metal coating 21 has multiple advantages. For one thing, translucent silicon dioxide can be used as the cover layer 12. This has advantages in the production of the measurement chip 1 (see below). It is true that the main layer 20 composed of silicon is essentially non-transparent for wavelengths of visible light up to 600 nm. However, silicon becomes increasingly more permeable to deep red and infrared light. This would be disruptive if the excitation light 80 or interference emissions 82 (not shown) lie in this wavelength range. However, titanium and gold are non-translucent far into the infrared wavelength range. As a result, shutters are formed by the coated cover layer 12, which partly block off excitation light 80 radiated in from the underside of the measurement chip 1 (not shown, see FIG. 2) when it exits from the measurement chamber 30. Likewise, interference emissions above the cover layer 12 are blocked off. The additional, non-translucent metal coating 21 therefore improves the measurement accuracy. Alternatively, the same effect can be achieved by means of a metal coating above the cover layer 12.

Another advantage is that the metal coating 21 can be contacted and used as an electrode for electrical measurements or excitations (not shown). The metal coating 21 can be used, in this manner, for characterization of the electrical properties of membranes 40, cell layers, or of transport systems situated in the membrane (not shown). In this connection, the measurement chip 1 can be used in such a manner that the impedance of a membrane 40 or cells spanned over it (not shown) can be measured. In this way, the tightness of membranes 40, cell layers, or tissue layers can be determined.

However, the measurement chip 1 can additionally be used also for production of an electrical field, particularly for control of voltage-sensitive transport systems, by means of the gold coating 21. These are, for example, voltage-dependent ion channels, i.e. ion channels that open or close at a specific limit value of the membrane voltage. By means of a change in the applied electrical field, functional switching processes that result in a change in the transport 70 of substrate molecules 60 by way of a membrane 40 can be triggered in this manner (not shown). The substrate molecules 60 can then be detected in the measurement chambers 30 by means of fluorescence.

A further use of the measurement chip 1 consists in that the upper cover layer 12 of the membrane chip 1 is covered with a lipid membrane that contains pore proteins, for example ion channels. An electrical field is applied to the electrode that acts as a gold coating 21 or an additional metallization on the measurement chip top 17 of the measurement chip, for a measurement. Another electrode in the solution above the membrane produces a membrane potential. The applied voltage leads to activation of the ion channels.

The embodiment of the measurement chip 1 as shown therefore has the advantage that biological transport systems can be switched in electrically functional manner and, at the same time, the transport by way of the membranes 40 produced in this manner can be measured optically, by means of fluorescence.

Yet another advantage occurs in combination with a chemically reactive or polar coating (not shown). Thiol compounds such as β-mercaptoethanol or mercaptopropionic acid, but also components of a detection system of the receptor activation can be bound to a gold coating 21 on the side wall 26 of the measurement chambers 30, in standardized manner. The main layer 20 composed of silicon, or the cover layer 12 composed of silicon oxide, does not bind these and therefore can be selectively modified by means of silanization. In this way, different modification of the side walls 26 of the measurement chambers 30 and of the top 17 of the measurement chip 1 is made possible, and this is advantageous for specific measurement tasks.

FIG. 5 furthermore shows the effect of the cover layer 12. Openings 14 of the cover layer 14 are disposed centered over the openings 25 of the measurement chambers 30. If a bundle of excitation light 80 is radiated in from below, then it is partly screened off by the cover layer 12 or reflected by the gold coating 21 on the underside 16 of the cover layer 12, and reaches the region above the measurement chip 1 only with reduced intensity, and this increases the measurement accuracy. Although interference emissions from above the measurement chip 1 penetrate the translucent cover layer 21 of silicon dioxide in the embodiment shown, they are reflected by the gold coating 21. FIG. 6 shows a vertical section through another embodiment of the measurement chip 1, having a main layer 20 composed of silicon, which has measurement chambers 30 in the shape of truncated cones. In the shape of truncated cones is understood to mean that the lower diameter 35 of the measurement chambers 30 decreases from the measurement chamber bottom 18 of the support layer 10 all the way to the upper measurement chamber opening 25. In this connection, excitation light 80 radiated in from the underside of the measurement chip 1 is better shielded against exiting from the upper opening 25 of the measurement chamber 30. FIG. 6 shows that excitation light 80 radiated in from below no longer leaves the measurement chamber opening 25, for the most part, because of the corrugated surface structure of the side walls 26 in combination with the measurement chamber 30 that narrows in an upward direction. Likewise, interference emissions 82 (not shown) above the main layer 20 are better shielded by the smaller upper opening 25. As the result of the synergistic effect of the two characteristics, the measurement accuracy that can be achieved with the measurement chip 1 is significantly increased even further.

FIG. 7 shows a top view of the measurement chip 1. In this connection, the drawing shows, in a partial view of the measurement chip 1, the measurement chambers 30, 30′, which are disposed in the form of an array. The measurement chambers 30 shown have a diameter 31 of 1 μm, but embodiments with diameters of a few nanometers up to multiple hundred micrometers are also possible. The distance 32 between the center points of the measurement chambers amounts to 2.5-4 μm, but a few nanometers are also possible.

The measurement chambers 30 have the basic shape of a circular cylinder. As FIG. 8 shows, however, the measurement chip 1 also has measurement chambers 30′ having a different shape, namely oval, in the top view shown and in cross-section. In this connection, an oval measurement chamber 30′ is provided periodically, in each instance, after a selected number, eleven in the measurement chip shown, of measurement chambers 30, specifically not only in the longitudinal but also in the transverse direction of the array. These measurement chambers 30′ serve as optical markings that can be recognized by the camera and allow simplified, clear assignment of the position of the measurement chambers 30 as well as manual or automated correction of lateral displacements of the measurement chip 1 during the measurements.

FIG. 8a shows a vertical section through a holder 200 for the microstructured measurement chip 1 described above. The holder 200 comprises a block-shaped plate having reservoirs 203 that can be filled through upper openings 205, preferably similar to commercially available microtiter plates, but also similar chambers in a length and width matching the microscope slide format. Preferably, the holder 200 also has the standardized height of a microtiter plate. In contrast to conventional microtiter plates, however, the reservoirs 203 are continuous channels, i.e. they do not have a bottom but rather a lower opening 210. The bottom of the reservoirs 203 is only formed by means of a measurement chip 1 attached to the underside 203 of the plate, for example, with a UV-curing adhesive, whereby the adhesive is cured by means of UV light after adjustment of the measurement chip 1. In this manner, the reservoirs 203, together with the microstructured measurement chip 1, form a chamber that can be filled with a desired measurement fluid.

In this connection, the measurement chip 1 is disposed in such a manner that its top 17 points toward the reservoirs 203 with the measurement chamber openings 25 (not shown), so that the measurement chambers 30 can be filled by the reservoirs 203. The optical measurement takes place from the underside 202 of the holder 200, through the lower, translucent support layer 10 of the measurement chip 1.

As FIG. 8a furthermore shows, the volume of the reservoirs 203 is increased in that their diameter increases in their lower section, in an upward direction, i.e. the diameter of the upper opening 205 of the reservoirs 203 is greater than the diameter of their lower opening 210, which is slightly smaller than the surface area of the measurement chips 1, so that these can be glued in place under the lower opening 210, forming a seal.

FIG. 8b shows a top view of the top 201 of the holder 200 from FIG. 9. The holder 200 has the length and width of a commercially available microscope slide. For example, 16 reservoirs 203 with measurement chips 1 glued under them are provided. The distance between the center points of the upper openings 205 amounts to 9 mm, and the diameter of the upper openings 205 amounts to 6 mm, whereby the diameter narrows in a downward direction, so that the lower opening 210 has a diameter of 2 mm. A square measurement chip 1 having a side length of 3 by 3 mm is glued under the lower opening 210.

FIG. 9 shows a vertical section through another preferred embodiment of the holder 200b. The holder 200b, like one shown in FIG. 8a, comprises a block-shaped plate having reservoirs 203 that can be filled through upper openings 205. However, the bottom of the reservoirs 203 is formed by a cover glass 215 having a thickness of about 50-200 μm. For this purpose, first all the measurement chips 1 are glued onto the cover glass 215 over their full area, using a non-fluorescent, transparent adhesive, preferably a silicone adhesive, particularly Sylguard 184. The entire cover glass 215 is then glued under the holder 200b, and seals off all the reservoirs 203, whereby the measurement chips 1 are countersunk into the reservoirs 203. In this manner, the reservoirs 203, together with the cover glass 215, form a chamber that can be filled with a desired measurement fluid. In order for the measurement chips 1 to find room in the lower openings 210, the lower opening 210 is slightly larger than in the case of the holder 200 shown in FIG. 8a. The cover glass 215 is translucent, so that the optical measurement can take place from below, through the cover glass 215.

FIG. 10 shows a vertical section through an SOI wafer 300 known according to the state of the art, in a partial view. According to the state of the art, it is used as a starting material or substrate for the production of electronic components and integrated circuits. In the present invention, however, it serves as a starting material or substrate for the production of the microstructured measurement chips 1. In the production according to the invention, known methods for the production of electronic components, such as photolithography and etching, are advantageously used.

The known SOI wafer 300 is composed of three layers firmly connected with one another, like a sandwich: a lower, thick, non-translucent silicon layer 311, an upper, thin, non-translucent silicon layer 320, and a very thin, so-called “buried” layer 312 (the English technical term is “buried layer”) disposed between them, which has electrically insulating properties and consists of translucent silicon dioxide.

The method for the production of the microstructured measurement chips 1 according to the invention from the SOI wafer 300 shown in FIG. 10 comprises essentially the following steps, which will be explained below, using FIGS. 11 to 15.

First, the depressions 30 that later serve as measurement chambers are introduced into the upper, thin, non-translucent silicon layer 320, by means of photolithography and suitable etching methods such as DRIE (Deep Reactive Ion Etching, Bosch process) or wet-chemical etching.

When using the Bosch process, alternating depressions 27 and elevations 28 form in the side walls 26, as a result of the alternating etching and passivation steps that are usual in this process, and these cause an essentially corrugated or riffled surface structure to form.

In etching, etching agents are used that dissolve only silicon, but not silicon dioxide. For this reason, etching advantageously takes place only to the buried silicon dioxide layer 312, which acts essentially as a “stop layer” and makes the etching process come to a stop. FIG. 11 shows the SOI wafer 300 from FIG. 10, with riffled measurement chambers 30 etched into the upper silicon layer 320. If desired, a metallization, for example of titanium or gold, can now be applied to the upper silicon layer.

As FIG. 12 shows, afterward the translucent support layer 10 composed of borosilicate glass is attached to the upper silicon layer 320, by means of anodic bonding.

Then, the lower silicon layer 311 is removed by means of etching, as FIG. 13 shows. During this method step, as well, etching advantageously takes place only to the buried silicon dioxide layer 312, which makes the etching process come to a stop.

FIG. 14 shows that the SOI wafer 300 worked on in this way is afterward turned over and is then in an “upside-down” position. As a result, the support layer 10 becomes the bottommost layer and the upper silicon layer 320 of the SOI wafer 300 becomes the later main layer 20 of the measurement chip 1. The originally buried layer 312 of the SOI wafer 300 is the uppermost layer and forms the later cover layer 12 of the measurement chip 1.

Afterward, the buried layer 312, which forms the cover layer 12 of the measurement chip 1, is partly structured or completely removed, photolithographically and with suitable etching methods, so that the openings 14, which act as shutters, are formed, which are preferably disposed centered above the measurement chambers 30. This is illustrated in FIG. 15, which corresponds to FIG. 4. The buried layer 312 can also be removed completely, thereby producing an embodiment of the measurement chip 1 as shown in FIG. 1.

Finally, individual measurement chips 1 are sawed out of the SOI wafer. The measurement chips 1 can be used individually or glued under the holder 200 described above, as shown in FIG. 8.

### REFERENCE SYMBOL LIST

1 measurement chip
5 vesicle
10 support layer
12 cover layer
14 cover layer opening
15 biological cell
16 cover layer underside
17 measurement chip top
18 measurement chamber bottom
20 main layer
21 coating
24 main layer underside
25 measurement chamber opening
27 measurement chamber side wall
28 depressions
30 elevations
30′ measurement chamber
30′ oval measurement chamber
31 measurement chamber diameter
32 distance between adjacent measurement chamber center points
33 measurement chamber depth
35 lower measurement chamber diameter
40 membrane
50 transporter molecule
60 substrate molecule
70 transport or diffusion through membrane
80 excitation light
81 emission
82 interference emission
90 membrane receptor
100 secreted protein
110 secretion
111 detection system
120 fluorescent molecule of a detection system
130 ligand
140 conversion to fluorescent molecule by detection system
200 holder
201 top
202 underside
203 reservoir
205 upper reservoir opening
210 lower reservoir opening
215 cover glass
230 control substrate
300 silicon wafer
311 lower silicon layer
312 buried layer
320 upper silicon layer

1. Microstructured measurement chip (1) for optical measurement of properties of artificial or biological membranes (40), having a lower, translucent support layer (10) and at least one essentially non-translucent main layer (20) disposed on top of the former, which layer has depressions (30) configured as measurement chambers, having an upper opening (25) and an inner side wall (26) or multiple inner side walls (26), wherein the side wall (26) or the side walls of the measurement chambers (30) have depressions (27) and/or elevations (28).

2. Microstructured measurement chip (1) according to claim 1, wherein the depressions (27) and elevations (28) alternate in the direction of the longitudinal axis of the measurement chambers (30) and the side wall (26) or the side walls have an essentially corrugated surface structure by means of the grooves formed in this way.

3. Microstructured measurement chip (1) according to claim 1, wherein the alternating depressions (27) and/or elevations (28) or grooves have a spacing of 0.1 to 0.6 μm and/or a depth of 20 to 110 nm.

4. Microstructured measurement chip (1) according to claim 1, wherein the measurement chambers (30) have the basic shape of a circular cylinder or a truncated cone, and the depressions (27) and elevations (28) that form the grooves run around the side wall (26) in circular shape.

5. Microstructured measurement chip (1) according to claim 1, wherein the lower, translucent support layer (10) consists of plastic or of glass, particularly of borosilicate glass, which is preferably produced according to the float method.

6. Microstructured measurement chip (1) according to claim 1, wherein the non-translucent main layer (20) demonstrates silicon or metal or plastic.

7. Microstructured measurement chip (1) according to claim 1, wherein a cover layer (12), preferably composed of silicon dioxide or silicon nitride, which has cover layer openings (14), the aperture of which is smaller than that of the openings (25) of the measurement chambers, above the openings (25) of the measurement chambers (30), is disposed on the main layer (20).

8. Microstructured measurement chip (1) according to claim 1, wherein its surface demonstrates one or more chemically reactive and/or polar coatings, particularly poly-L-lysine and/or propionic acid and/or carboxyl groups and/or lipid derivatives and/or amino-reactive linker molecules, in order to bind suitable components of a membrane or of the transport system to the measurement chip (1) covalently or non-covalently.

9. Microstructured measurement chip (1) according to claim 1, wherein the side wall or the side walls (26) of the measurement chambers (30) and/or the underside of the main layer (20) that faces the support layer (10) and/or the underside of the cover layer (12) that faces the measurement chambers (30) and/or the top of the main layer (20) have an additional non-translucent coating (21), in each instance, preferably composed of metal, particularly of gold or titanium.

10. Microstructured measurement chip (1) according to claim 1, wherein the ratio of depth (33) to diameter (31) of the measurement chambers (30) is greater than one, preferably greater than five, and particularly preferably ten to fifty.

11. Microstructured measurement chip (1) according to claim 1, wherein the measurement chambers (30) are disposed as an array and preferably have optical markings, which are particularly configured as oval-shaped measurement chambers (30′) or measurement chamber openings (25).

12. Holder (200) for microstructured measurement chips (1) according to claim 1, wherein the holder (200) comprises a plate having a top (201), an underside (202), and one or more reservoirs (203) that can be filled with fluid from the top, and the bottom of a reservoir (201), in each instance, is formed by a measurement chip attached to the underside (202) of the plate (200), in each instance, or a cover glass with measurement chips (1) glued onto it.

13. Method for the production of microstructured measurement chips (1) for optical measurement of properties of artificial or biological membranes (40), according to claim 1, wherein a wafer (300) that demonstrates silicon is used as the substrate.

14. Method according to claim 13, wherein a silicon wafer is used, which has a lower silicon layer (311), an upper silicon layer (320), and a buried layer (312) disposed between them, and, in particular, is a silicon on insulator wafer, and the buried layer (312) preferably consists of silicon dioxide or silicon nitride.

15. Method according to claim 14, wherein the measurement chambers (30) are etched into the upper silicon layer (320) all the way to the buried layer (312), preferably using the DRIE method.

16. Method according to claim 13, wherein a non-translucent coating (21), is applied to the silicon wafer (300).

17. Method according to claim 14, wherein the upper silicon layer (320) of the silicon wafer (300), having the measurement chambers (30) etched into it, is connected with the support layer (10), preferably by means of anodic bonding.

18. Method according to claim 14, wherein the lower silicon layer (311) of the silicon wafer (300) is removed, preferably by means of etching.

19. Method according to claim 14, wherein the buried layer (312) is removed, preferably by means of etching, completely or selectively at the locations that cover the measurement chambers (30), so that cover layer openings (14), are formed.

20. Method according to claim 14, wherein individual microstructured measurement chips (1) are sawed out of the silicon wafer (300), and are particularly glued onto the holder (200).
